(19) United States
(12) Patent Application Publication
(10) Pub. No.: US 2018/0213835 A1
(43) Pub. Date: Aug. 2, 2018

(54) VEGETABLE HEALTH DRINK

(71) Applicant: Oatly AB, MALMÖ (SE)

(72) Inventor: Angeliki TRIANTAFYLLOU, Alimos (GR)

(21) Appl. No.: 15/748,371

(22) PCT Filed: Jul. 29, 2016
(86) PCT No.: PCT/SE2016/000040

§ 371 Date: Jan. 29, 2018

(30) Foreign Application Priority Data
Jul. 30, 2015 (SE) 1500321-3
Apr. 5, 2016 (SE) 1600124-0

(51) Int. Cl. A23L 33/185; A23L 2/66; A23L 2/84; A23L 33/125; A61K 9/00; A61K 38/01

(52) U.S. Cl. CPC A23L 33/185; A23L 2/66; A23L 2/84; A23V 2002/00; A61K 9/0095; A61K 38/011; A23L 33/125

(57) ABSTRACT

An oat based vegetable health drink comprises or substantially consists of carbohydrate of high glycemic index (GI) from oats, inherent oat protein, β-glucan, potato protein or other protein of vegetable origin of a composition similar to that of potato protein, and water but does not contain any of: protein of animal origin, trimethylglycine, hydrocolloid other than β-glucan. Also disclosed is a method of promoting muscle glycogen recovery upon physical exertion by administration of a glycogen recovery efficient amount of the health drink in close timely proximity of the exertion.

### FIELD OF THE INVENTION

The present invention relates to a vegetable oat based health drink and to a method of promoting muscle glycogen recovery or of delaying physical exertion by administration of the drink.

### BACKGROUND OF THE INVENTION

A large number of healthy nutrition drinks are presently on the market. Some of them are designed for use in connection with physical exercise. Their aim is to increase physical performance rather than health. For fast recovery after exercise there is a need to replenish depleted glycogen stores from the carbohydrate content of the drink. Carbohydrates of such drinks are of a high glycemic index. After both aerobe and anaerobe exercise insulin sensitivity is temporarily increased enabling a higher rate of glycogen synthesis. The protein content of a health drink, if any, augments the effect by increasing insulin release.

Currently available drinks of this kind are usually milk based, their main protein component being whey protein or casein. A high intake of milk and animal based protein however increases the risk of cardiovascular disease. Another problem with drinks comprising animal based protein is their allergenic nature. On the other hand protein of vegetable origin is considered inferior to protein from animal sources in respect of composition and thus nutritional value.

Methods for isolation of total potato protein as well as its major constituents patatin (40-50% by weight), protease inhibitors (30-40% by weight) and other proteins (10-20% by weight) is disclosed in WO 2008/069650 A1. WO 2014/011042 A1 discloses a method for isolation of potato protein constituents by absorption on a polymeric material comprising suitable ligands. WO 2011/059330 A1 discloses a potato protein glycated with a reducing sugar for use as a food additive.

### OBJECTS OF THE INVENTION

One object of the invention is to provide a vegetable health drink that is free from the drawbacks of health drinks comprising protein from animal sources while being at least as efficient in respect of muscle glycogen recovery upon physical exertion.

Further objects of the invention will become evident from a study of the following summary of the invention, the description of preferred embodiments thereof and the appended claims.

### SUMMARY OF THE INVENTION

According to the present invention is disclosed a vegetable health drink comprising or substantially consisting of carbohydrate of high glycemic index (GI), β-glucan, oat protein, potato protein or other vegetable protein of a composition similar to that of potato protein, and water but which does not contain any of: protein of animal origin, trimethylglycine, hydrocolloid other than β-glucan. The carbohydrate of high GI is preferably a mixture of maltose and low molecular weight oligosaccharides containing little or no glucose, in particular less than 1% or 2% by weight of total solids.

It is preferred for the carbohydrate of high GI to be a mixture of disaccharides, in particular maltose, and low-molecular weight oligosaccharides obtained or obtainable by enzymatic degradation of an oats source such as oat meal, in particular an oat source of which the β-glucanase content has been substantially reduced, such as to an extent of 80% or more or 90% or more or 95% or more or eliminated. A preferred method of β-glucanase content reduction or elimination is heat treatment of the oats source. This process also releases oat protein contained in the oats source, which protein is termed inherent oat protein in this application. The content of inherent oat protein in the vegetable health drink of the invention is about 1% by weight of dry matter. Raising the protein content of the drink requires addition of protein from vegetable sources other than oat. The vegetable health drink of the invention may additionally comprise added glucose and/or sucrose but this is not preferred. It is preferred for the β-glucan of the vegetable health drink to be obtained or obtainable by enzymatic degradation of the starch component of 5 said oats source, in particular with a-amylase or β-amylase or a combination thereof. Low-molecular oligosaccharides of the invention particularly comprise or consist of oligosaccharides of a molecular weight of less than 5000, in particular of less than 3000.

According to preferred aspect of the invention the health drink of the invention is based on an oat drink obtained or obtainable by a process disclosed in EP 1383396 A, EP 1123012 A or WO 2014/123466 A1, which is enriched with a protein of vegetable origin and which does not contain protein of animal origin.

A preferred protein of vegetable origin is one containing:

| amino acid | % by weight |
|---|---|
| Ile | 4.0-7.0, |
| Leu | 7.0-10.0, |
| Val | 4.0-9.0, |
| Lys | 5.0-8.0, |
| Met | 0.5-2.0, |
| Phe | 5.0-8.0, |
| Thr | 3.0-6.0, |
| Trp | 0.5-2.0, |
| His | 1.0-3.0, |
| Cys | 1.0-3.0, |
| Tyr | 4.0-6.0, |
| Ala | 1.0-3.0, |
| Arg | 3.0-6.0, |
| Asp + Asn | 10-15, |
| Glu + Gln | 6.0-9.0, |
| Gly | 4.0-7.0, |
| Pro | 4.0-6.0, |
| Ser | 4.0-6.0, |
| the total adding up to | 100%. |

A particularly preferred protein of vegetable origin is potato protein. Another particularly preferred protein of vegetable origin is potato protein glycated with a reducing mono- or di-saccharide or dextran or combinations thereof.

A preferred health drink of the invention comprises in % by weight:
about 1% of inherent oat protein;
from 2% to 5% of potato protein;
from 0.1% to 0.4%, particular from 0.2%-0.3% of beta glucan;
from 0,5% till 4,0% of vegetable oil;
from 4% to 12% of maltose and low molecular weight polysaccharide;
water up to 100%

The preferred health drink of the invention can optionally comprise one or more of:
from 2% to 6% of vegetable oil;
from 0% to 2% of dextrose;
calcium salt selected from tri-calcium phosphate, di-calcium phosphate, calcium carbonate;
sodium chloride;
aroma.

The vegetable health drink of the invention is at least as efficient as or more efficient in respect of muscle glycogen recovery upon physical exertion than a health drink of same composition comprising protein from an animal source in the same amount as vegetable protein, in particular at least as efficient as or more efficient than a health drink of same composition comprising milk protein in the same amount as vegetable protein. The muscle glycogen recovery upon physical exertion may be intake of a muscle glycogen recovery effective amount of the vegetable health drink of the invention prior to physical exertion or post physical exertion. To be effective, the health drink should be taken within a short period prior to the start of exertion or within a short period after the end of exertion but may also be taken during exertion. Said short period is a period of 30 min, in particular of 15 min, most particularly of 10 min or 5 min prior or post exertion. A possible but not binding explanation for the efficacy of the health drink of the invention is its rapid transfer from the stomach to the small intestine, stomach emptying being accelerated by the low viscosity of the drink. Oat based health drinks enriched by corresponding amounts of other vegetable proteins such as pea protein and lentil protein are substantially more viscous and thus less prone to be rapidly emptied from the stomach.

According to an important aspect the health drink of the invention is substantially more efficient in delaying physical exertion than a health drink of same composition comprising dairy milk protein instead of oat protein, potato protein and/or other protein of vegetable origin.

According to the invention is furthermore disclosed a method of promoting muscle glycogen recovery upon physical exertion or of delaying physical exertion, comprising administration of a glycogen recovery efficient amount or physical exertion delaying amount of the vegetable health drink of the invention in close timely proximity of the physical exertion. In the method administration can occur prior to the start of the activity resulting in physical exertion or after the end of the activity or during activity. In the method a glycogen recovery or physically exertion delaying efficient amount is >100 ml, in particular >200 ml, such as 250 ml or more of the drink of the invention.

The vegetable health drink of the invention can be manufactured by providing an oat base of the aforementioned kind in a container and admixing the other constituents to the oat base while stirring. The vegetable health drink is then pasteurized and packed aseptically. From a consumer's standpoint the provision of health drinks of about neutral pH, such as chocolate drinks, and of acidic pH, such as fruit drinks, is desirable.

The invention will now be explained in more detail by reference to a number of preferred embodiments thereof illustrated by a number of figures.

### DESCRIPTION OF THE FIGURES

FIG. 1 is a diagram showing the viscosity of protein-enriched oat based drinks at pH 6.5;

FIG. 2 is a diagram showing the viscosity of protein-enriched oat based drinks at pH 6.5;

FIG. 3 is a diagram showing insulin levels (μIU/mL) during an endurance test followed by a recovery period.

### DESCRIPTION OF PREFERRED EMBODIMENTS

Potato protein for use in the invention is available from AVEBE U.A., Veendam, Netherlands under the trade name Solanic™. Another supplier of potato protein is Karup Kartoffelfabrik A.m.b.A., Karup, Denmark. Beta-glucan for use in the invention (other than as a natural component of oat base) is available from Tate & Lyle Sweden AB, Kimstad, Sweden.

Examples 1-3 describe drinks for use in a clinical study. The drink in Example 1 is a drink according to the invention whereas the drinks of Examples 2 and 3 are for comparison only. The drinks of Examples 1-3 are calorie equivalent.

### EXAMPLE 1

### Vegetable Health Drink

A vegetable health drink of the invention was prepared by mixing the ingredients listed in Table 1 at room temperature.

The drink was compared in respect of muscle glycogen recovery upon physical exertion with the drinks of EXAMPLES 2 and 3.

TABLE 1
Vegetable health drink A of the invention

| Ingredient | g/100 g (% by weight) |
|---|---|
| Oat base, 11.5% dry matter | 66.520 |
| Water | 27.500 |
| Potato protein Solanic ® 300XP | 2.400 |
| Potato protein Solanic ® 300N | 1.200 |
| Rapeseed oil | 0.900 |
| Vanilla aroma | 0.200 |
| NaCl | 0.100 |
| Tri-calcium phosphate | 0.094 |
| Di-calcium phosphate | 0.047 |
| Calcium carbonate | 0.188 |
| Commercial fibre composition* | 1.000 |
| Total | 100.000 |

*PromOat ™ Beta Glucan, TATE & LYLE Oat Ingredients, Kimstad, Sweden. Nutritional data per 100 g: Energy 315 kcal/1319 kJ; fat 0.5 g; oat maltodextrin 56 g containing less than 0.5 g sugars; fibre (oat β-glucan 35 g; protein 4 g; salt <70 mg.

### EXAMPLE 2

### Milk Drink of Same Carbohydrate, Protein, and Fat Content as the Health Drink of Example 1

For comparison with the health drink of the invention of Example 1 a milk drink was prepared by mixing the components listed in Table 2 at room temperature.

TABLE 2
Dairy milk drink

| Ingredient | g/100 g (% by weight) |
|---|---|
| Low lactose dairy milk | 97.050 |
| Rapeseed oil | 0.500 |
| Whey | 0.150 |
| Casein | 0.600 |
| Dextrose | 1.500 |
| Vanilla aroma | 0.200 |
| NaCl | 0.100 |
| Total | 100.000 |

### EXAMPLE 3

### Maltodextrin Drink, Negative Control

For comparison with the health drink of the invention of Example 1 a maltodextrin drink was prepared by mixing the components listed in Table 3. It has the same energy content per volume as the potato protein drink of the invention of Example 1 and the low lactose milk drinks of Example 2 but does not comprise protein.

TABLE 3
Maltodextrin drink

| Ingredient | g/100 g (% by weight) |
|---|---|
| Water | 90.500 |
| Maltodextrin | 3.800 |
| Dextrose | 2.000 |
| Rapeseed oil | 3.000 |
| Vanilla aroma | 0.200 |
| Salt | 0.100 |
| Total | 100.000 |

### EXAMPLE 4

### Comparative Muscle Glycogen Recovery Study

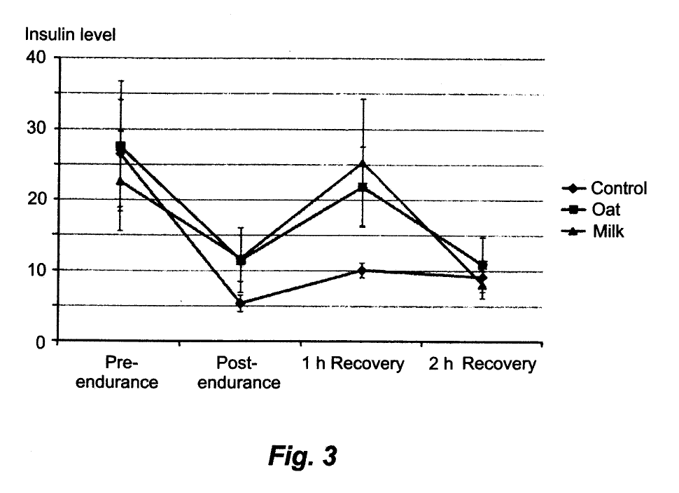

Exercise experiments performed on slightly active and overweight male subjects, age 20-40 years, BMI from 28 to 32.
i) Pre-intervention test: Registration of 3 day food record, energy expenditure measurement by Actiheart™ device. Whole body DEXA scan performed on day 1 to determine body composition. Collection of blood samples and biopsy samples from vastus lateralis muscle. Test drink volume of 250 ml per consumption.
ii) Initial exercise tests on separate days:
   a. Subject matching. Matching according to VO₂max and training experience; subjects randomized into one of three groups: Oat based drink according to the invention, milk based drink, maltodextrin drink (control);
   b. First endurance test. Test drinks consumed 15 min prior to and directly after an endurance test at 70% VO₂max for 90 min. Collection of blood samples before first drink, directly upon exercise, and after recovery periods of 60 min and 120 min (FIG. 3);
   c. Second endurance test. Performed at 80% VO₂max until exhaustion. Test drinks consumed 15 min prior to exercise and directly after. Collection of blood samples before first drink, directly upon exercise, and after recovery periods of 60 min and 120 min.
iii) Intervention period of 6 weeks' physical training comprising 3-4 weekly supervised and controlled biking and running sessions. Training load 60%-80% VO₂max; training session length from 45 min to 90 min. Heart rate monitoring during sessions and adapting load accordingly. Nutrition and diet according to food intake guidelines. No food or other energy containing drink intake within 2 h from intake of test drink. The tests furthermore indicated that the drink according to the invention (Example 1) was as efficient and even better in regard of muscle glycogen recovery upon physical exertion as a health drink of same energy content and composition comprising protein from an animal source (Example 2) and more efficient than a drink of same energy content lacking protein (Example 3). During recovery after the endurance test the drink according to the invention produced significantly increased insulin levels at 1 h recovery in comparison with those produced by dairy milk.
iv) Body composition, blood samples and VO₂max were determined after intervention and the endurance tests repeated by following the same protocols as before intervention.

Table 3a illustrates the time consumed until physical exertion in the first endurance test. The drink according to the invention substantially extended the time period until physical exertion.

TABLE 3a
Time to physical exertion in first performance test

| | Number of | Time (min), | Std. | |
|---|---|---|---|---|
| Group | Participants | mean | Deviation | Std. Error |
| Control | 10 | 19.31 | 9.24 | 2.92 |
| Oat drink | 8 | 22.67 | 11.88 | 4.19 |
| Dairy milk | 10 | 11.36 | 3.41 | 3.42 |

### EXAMPLE 5

### Vegetable Health Drink B of the Invention—Apple/Green Tea/Spinach

To an oat base prepared by two-step enzymatic degradation with α-amylase and β-amylase from a suspension of 578 kg heat-treated oat meal in water (4170 kg) was admixed under stirring at 10 about 55° C. to 60° C. potato protein (Solanic™ 300XP, 125 kg, then Solanic™ 300N, 45 kg) followed by admixture of 750 kg of a mixture of apple juice, green tea and spinach juice. The product had a pH of about 6.3. Aqueous solutions of the commercially available potato proteins Solanic™ 300XP and Solanic™ 300N differ slightly in their pH; their mixture can varied to adjust the pH of the final product, here to pH 4.1±0.2, which is then pasteurized at about 105° C., cooled, and packed aseptically in sterile containers. The vegetable health drink so produced has a viscosity (sp2/60 rpm/25° C.±2° C. of <50 cP (0.05 Pa·s). The vegetable health drinks of the invention of a pH of 6-7 can be pasteurized at a temperature of about 140° C. for 4 sec-8 sec, and are then storage stable at room temperature if filled aseptically into TetraPak™ paper board containers. Drinks of a pH of about 5 can be pasteurized at a temperature of about 125° C. for storage at room temperature. It is preferable to use a pasteurization temperature of about 105° C. for slightly acidic drinks to be stored at refrigerator temperatures (about 5° C.)

### EXAMPLE 6

### Vegetable Health Drink C of the Invention—Red Beets/Ginger

To an oat base prepared by two-step enzymatic degradation with α-amylase and β-amylase from a suspension of 578 kg heat-treated oat meal in water (4170 kg) was admixed under stirring at room temperature potato protein (Solanic™ 300XP, 125 kg, then Solanic™ 300N, 45 kg) followed by admixture of 600 kg of a mixture of a juice prepared by grinding red beets and ginger, and filtration of the obtained slurry. The mixture obtained after addition of red beets/ginger juice has a pH of about 6.3. Aqueous solutions of the commercially available potato proteins Solanic™ 300XP and Solanic™ 300N differ slightly in their pH; their mixture can varied to adjust the pH of the final product to pH 4.1±0.2 in the same manner as in Example 5. The vegetable health drink had a viscosity (sp2/60 rpm/25° C.±2° C. of <50 cP (0.05 Pa·s).

### EXAMPLE 7

### Shear Viscosity of Protein-Enriched Oat Drinks

Protein-enriched oat drinks of a final protein content of 5% by weight of dry matter were prepared by adding different vegetable protein isolates to oat drink (Oat drink containing 1 by weight of vegetable protein, that is, inherent oat protein stemming from the oats source from which the drink was prepared). Since all samples contain inherent (non-added) oat protein from the oat source of the drink reference to the inherent oat protein will be omitted in the following. The protein isolate sources included potato (Solanic™ 300N and Solanic™ 300XP, respectively), lentil (Ingredion Vitessence™ Pulse 2550), and pea (Nutralys™ S85F).

Sample preparation. The formulations of in Table 4 were immersed in a boiling water bath, the temperature of the bottle contents being controlled by thermometers. The bottles were removed 5 min after their content had reached 90° C., and cooled to room temperature by immersion in cold water. The content of each bottle was divided in half by transfer into pairs of glass tubes, the pH of which was adjusted by 5 M HCl and 2 M NaOH to 6.5 and 3.5, corr. to pH in a neutral drink as well with added fruit puree.

TABLE 4
Protein-enriched oat drink, total protein
content of 5% by weight

| | | | Formulation | | |
|---|---|---|---|---|---|
| Added protein isolate | | | Protein | Mixed | |
| | | Protein | isolate | with | Formulation, |
| | | content | added, | g of Oatly | total weight |
| Source | Brand | g/100 g | g | blue | (g) |
| Potato | Solanic ™ 300N | 93 | 4.3 | 95.7 | 100 |
| Potato | Solanic ™ 300XP | 93 | 4.3 | 95.7 | 100 |
| Lentil | Vitessence ™ | 55 | 7.3 | 92.7 | 100 |
| | 2550 | | | | |
| Pea | Nutralys ™ S85F | 80 | 5.0 | 95.0 | 100 |

Viscosity measurements. Shear viscosity was measured with a rheometer with bob-cup geometry (Kinexus range, Malvern Instruments, UK). Settings included viscosity as a function of shear rate at 25° C., 7 points per decade. Samples where sediment was observed were gently re-suspended by slow stirring with a spoon and allowed to rest for 5 min before measurement. Values reported are shear viscosity as well as consistency index (m) and behavior index (n) calculated using the power-law model in the range of 0.1 to 20 s⁻¹. A behavior index of 1 indicates Newtonian behavior, one of <1 shear thinning, one of >1 shear thickening.

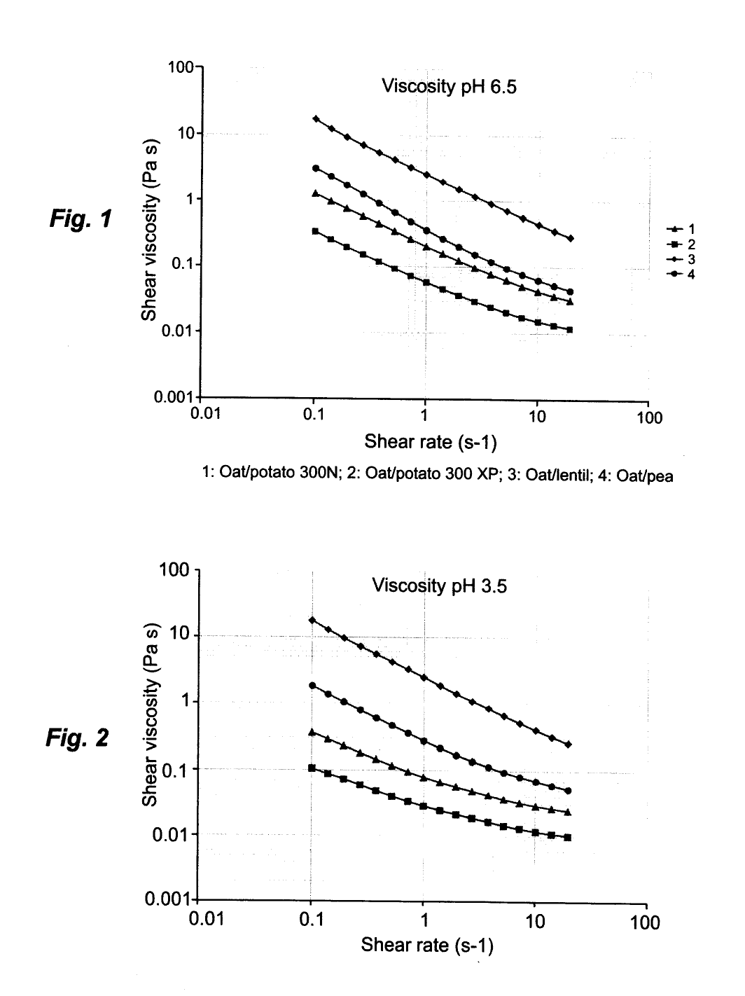

Results: Samples of pH 6.5. Shear viscosity (Pa·s) over a shear rate range of from 0.1 s⁻¹ to 20 s⁻¹ is shown in FIG. 1. Potato had the lowest viscosity, followed by pea and lentil. The samples behaved like Newtonian fluids of shear rate independent viscosity. Consistency index (m) and behavior index (n-1) were calculated using the power-law model (Table 5).

Results: Samples of pH 3.5. Potato had the lowest viscosity, followed by pea and lentil (FIG. 2). The consistency index followed the same pattern, all samples displaying shear thinning behavior (Table 5).

TABLE 5
Power law model based average consistency index (m) and
behavior index (n) values of oat drinks at pH 6.5 and 3.5

| | | | |
|---|---|---|---|
| Sample | m | n | R² |

pH 6.5

| | | | |
|---|---|---|---|
| Oat/potato 300N | 0.219 | 0.279 | 0.995 |
| Oat/potato 300XP | 0.064 | 0.348 | 0.989 |
| Oat/lentil | 2.615 | 0.231 | 0.999 |
| Oat/pea | 0.407 | 0.169 | 0.993 |

pH 3.5

| | | | |
|---|---|---|---|
| Oat/potato 300N | 0.090 | 0.482 | 0.982 |
| Oat/potato 300XP | 0.032 | 0.546 | 0.980 |
| Oat/lentil | 0.312 | 0.318 | 0.987 |
| Oat/pea | 2.522 | 0.194 | 0.999 |

1. Oat-based vegetable health drink comprising or substantially consisting of carbohydrate of high glycemic index (GI) , inherent oat protein, β-glucan, potato protein or other protein of vegetable origin of a composition similar to that of potato protein, and water but which does not contain any of: protein of animal origin, trimethylglycine , hydrocolloid other than β-glucan, wherein the carbohydrate of high GI is a mixture of disaccharides, in particular maltose, and low-molecular weight oligosaccharides obtained or obtainable by enzymatic degradation of an oats source.

2. The drink of claim 1, wherein the carbohydrate of high GI is a mixture of maltose and of low molecular weight oligosaccharides containing little or no glucose.

3. The drink of claim 2, containing glucose in an amount of less than 2% by weight of total solids.

4. The drink of claim 2, containing glucose in an amount of less than 1% by weight of total solids.

5. The drink of claim 1,

6. The drink of claim 5, wherein the oats source is oat meal of which the β-glucanase content has been substantially reduced, such as to less than 20% or less than 10% or less than 5% of the original content, or eliminated.

7. The drink of claim 6, wherein the β-glucanase content of the oats source has been reduced or eliminated by heat treatment.

8. The drink of claim 1, wherein the β-glucan of the drink has been obtained by enzymatic degradation of the starch component of said oats source.

9. The drink of claim 8, wherein a-amylase or β-amylase or a combination thereof have been used for degradation of starch.

10. The drink of claim 1, wherein in the vegetable protein is one containing by weight: lie, 4.0-7.0; Leu, 7.0-10.0 Val, 4.0-9.0; Lys, 5.0-8.0; Met 0.5-2.0; Phe, 5.0-8.0; Thr 3.0-6.0; Trp 0.5-2.0; His 1.0-3.0; Cys 1.0-3.0; Tyr 4.0-6.0; Ala 1.0 3.0; Arg 3.0-6.0; Asp +Asn, 10-15; Glu +Gin; 6.0-9.0; Gly 4.0-7.0; Pro 4.0-6.0; Ser 4.0-6.0; the total adding up to 100%.

11. The drink of claim 1, wherein protein of vegetable origin is potato protein.

12. The drink claim 1, wherein protein of vegetable origin is potato protein glycated with a reducing mono or di-saccharide or dextran or combinations thereof.

13. Vegetable health drink comprising, in % by weight:
from 2% to 5% of potato protein;
from 0.1° A to 0.4%, in particular from 0.2%-0.3% beta glucan;
from 0,5% till 4,0% and up to 6% of vegetable oil;
from 4% to 12% of maltose and low molecular weight polysaccharide;
water up to 100%;
but not comprising glucose in amount of >2% by weight.

14. The vegetable health drink of claim 13, comprising
from 2% to 6% of vegetable oil;
from 0% to 2% of dextrose;
calcium salt selected from tri-calcium phosphate, di-calcium phosphate, calcium carbonate;
sodium chloride;
aroma.

15. A method of promoting muscle glycogen recovery upon physical exertion or of delaying physical exertion, comprising administration of a glycogen recovery efficient or physical exertion delaying amount of the health drink of claim 1 in close timely proximity of the physical exertion.

16. The method of claim 15, wherein administration occurs prior to the start of the activity resulting in physical exertion.

17. The method of claim 15, wherein administration occurs after the end of the activity resulting in physical exertion.

18. The method of claim 15, wherein administration occurs during the activity resulting in physical exertion.

19. The method of claim 15, wherein a glycogen recovery efficient or physical exertion delaying amount is >100 ml, in particular >200, such as 250 ml or more of the drink of claim 1.
